(19) United States
(12) Patent Application Publication
(10) Pub. No.: US 2012/0028329 A1
(43) Pub. Date: Feb. 2, 2012

(54) OPTICALLY TRANSPARENT GLASS AND GLASS-CERAMIC FOAMS, METHOD FOR PRODUCTION THEREOF AND USE THEREOF

(76) Inventors: Michael Scheffler, Magdeburg (DE); Christiane Ohl, Cottbus (DE); Christina Olschewski, Glienicke (DE); Viola Wilker, Cottbus (DE); Franziska Scheffler, Magdeburg (DE)

(21) Appl. No.: 13/138,200

(22) PCT Filed: Jun. 24, 2009
(86) PCT No.: PCT/EP2009/057866

§ 371 Date: Oct. 6, 2011

(30) Foreign Application Priority Data
Jan. 16, 2009 (EP) 09150767.3

(51) Int. Cl. C12N 9/00; C12N 1/00; B01J 32/00; C03B 19/08; C03C 11/00

(52) U.S. Cl. 435/183; 65/22; 501/39; 501/32; 502/439; 435/243

(57) ABSTRACT

The present invention relates to an optically transparent glass foam or glass-ceramic foam as well as to a method for the production of an optically transparent glass foam or glass-ceramic foam. A method is described for the production of optically transparent foams, wherein the following steps are conducted:

a) Mixing of pre-ceramic Si polymer, glass powder and glass converter;
b) Heating of the mixture to a temperature below the decomposition temperature of the pre-ceramic Si polymer with the formation of a foam;
c) Heating the obtained foam to a temperature above the decomposition temperature of the pre-ceramic Si polymer;
d) Heating the obtained product to a temperature between 700° C. and 1400° C. with the formation of glass; and
e) Cooling the thus-obtained product to ambient temperature.

In addition, a use for predominantly optical applications is described, such as, for example, as a support for photocatalysts, a support for enzymes/microbes that operate with light/sunlight in bioreactors, in environmental catalysis, biocatalysis and, for example, for removal of VOCs from wastewater and air.

The invention relates to optically transparent foams, a method for the production thereof and use thereof.

### PRIOR ART

### Relevant Methods for the Production of Glass Foams

Methods for the production of glass foams are already known in the prior art. Thus, for example, glass foams and the production of glass foams are described by G. Scarinci et al. (G. Scarinci, G. Brusatin, E. Bernardo: Glass Foams. In: M. Scheffler, P. Colombo (Editors): Cellular Ceramics Structure, Manufacturing, Properties and Applications. 158-176, Wiley-VCH Publishers GmbH Co KGaA, Weinheim, ISBN: 3-527-31320-6, 2005) and L. Kern in U.S. Pat. No. 1,898,839, filed in June 1930. The glass foams produced by means of the disclosed methods are not suitable, however, for optical applications. The criterion for use in optical applications is a high light transmission of the material obtained.

M. Frosch, Diploma Thesis, University of Erlangen, 2007, proposes a method for the production of a glass foam based on SiO₂. In this case, a polymer foam such as is described by K. Schwartzwalder and A. V. Somers in U.S. Pat. No. 3,090,094, filed in February 1961, is coated with a polysiloxane and quartz glass powder as a filler. In a complicated thermal process, which is carried out stepwise at temperatures up to 1600° C., there is produced the fritting of the organic template sponge and the sintering process of the silicon dioxide that forms by the oxidation of the polysiloxane to quartz glass or to a quartz glass/cristobalite mixture. The material is characterized by an average optical transparency. The high sintering temperatures, however, act as a disadvantage on obtaining the macroporous foam structure and low mechanical strengths are produced.

In addition, the use of glass powder with a lower glass transition range than quartz glass as a filler in the production of glass foams is described by J. D. Torrey (presented at the 32nd International Conference & Exposition on Advanced Ceramics and Composites, Daytona Beach, Fla., USA, Jan. 27, 2008-Feb. 1, 2008). Duranglas® was used as the glass filling powder. In this method, however, it is a disadvantage that two glass phases with different compositions form or remain present next to one another and the light transmission is reduced due to the difference in the refractive index of the two glasses.

### Method for the Production of Polymer-Derived Ceramic Foams

Polymer-derived ceramic foams are produced according to a plurality of methods that are the subject of numerous publications and technical papers, such as, for example, Bao, X., Nangrejo, M. R. & Edirisinghe, M. J., J. Mater. Sci., 1999, 34, 2495-2505; Gambaryan-Roisman, T., Scheffler, M., Buhler, P., Greil, P., Ceram. Trans., 2000, 108, 121-130; Colombo, P., Bernardo, E., Comp. Sci. Tech., 2003a, 63, 2353-2359; Scheffler M. and Colombo P. (eds.), Cellular Ceramics: Structure, Manufacturing, Properties and Applications, WILEY-VCH Weinheim, Germany, 2005 and Zeschky, J., Ceramic foams of filled polysilsesquioxanes, Dissertation, University of Erlangen, 2004.

In the course of this method, a so-called green foam is produced first by mixing a pre-ceramic polymer, preferably a polysiloxane, with a filler, and subsequently either foaming this green foam directly or converting it into a cross-linked thermoset that cannot be melted by a molding process (see Colombo, P., Bernardo, E., Comp. Sci. Tech., 2003a, 63, 2353-2359; Gambaryan-Roisman, T., Scheffler, M., Buhler, P., Greil, P., Ceram. Trans., 2000, 108, 121-130; K. Schwartzwalder, A. V. Somers, U.S. Pat. No. 3,090,094).

In connection with the molding/foaming, the thermoset obtained in this way is thermally converted to the so-called polymer-derived ceramic foam in an inert or reactive atmosphere.

There is an urgent need, however, for glass foams and glass-ceramic foams with high light transmission. Catalysts that can be activated by means of light, so-called photocatalysts, are used in a number of applications in the chemical industry and in environmental catalysis. If these types of catalysts are provided for the acceleration of reactions in liquid or gaseous phase or even in solid form, fixation onto a support is necessary. High requirements are placed on this type of support relative to its transmission of light with the wavelengths activating the catalyst, but there are also requirements relative to the permeability of flowing liquids and gases. Monolithic glass and ceramic structures such as open-cell foams and honeycomb structures are suitable for this purpose.

### PROBLEM OF THE INVENTION

The problem of the present invention is to overcome the above-described disadvantages of the prior art.

In particular, it is a problem of the present invention to provide a cellular support, which is equipped with good permeability for gases and liquids and a high optical transparency for a large part of the light of wavelengths of the solar spectrum. It is preferably a glass having a high silicate fraction of conventional composition.

### DESCRIPTION OF THE INVENTION

The problem of the invention is solved by providing an optically transparent glass foam or glass-ceramic foam, which can be produced by the method of the invention according to claim 1.

In order to overcome the problems of the prior art described above and to fine-tune the optical properties, a method was developed in which a glass phase with a composition similar to or the same as that of the filler glass phase forms directly during the thermal conversion of a foamed, organosilicon polymer batch filled with glass.

The problem is thus solved by a method as described in claim 1. Advantageous embodiments of the method according to the invention are characterized in the dependent subclaims.

The subject of the invention is a method for the production of optically transparent foams, wherein the following steps are conducted.

a) Mixing of pre-ceramic Si polymers, glass powders and glass converters;
b) Heating of the mixture to a temperature below the decomposition temperature of the pre-ceramic Si polymer with the formation of a foam;
c) Heating the obtained foam to a temperature above the decomposition temperature of the pre-ceramic Si polymer;
d) Heating the obtained product to a temperature between 700° C. and 1400° C. with the formation of glass; and
e) Cooling the thus-obtained product to ambient temperature.

In this case, a method is preferred in which an additional step f) is conducted after step a), comprising:

f) Introducing and/or applying the mixture produced in step a) onto and/or into an organic polymer foam material and subsequently drying the material optionally at elevated temperature.

Further, a method is preferred wherein step c) is conducted at a temperature of 350° C. to 750° C.

A method is particularly preferred wherein step d) is conducted at a temperature of less than 1100° C.

In addition, a method is preferred in which the heating in step d) is conducted for 1 to 12 hours at maximum temperature.

A method is also preferred wherein the product obtained in step e) is rapidly cooled by removal from the oven.

A method is most particularly preferred wherein the product obtained in step e) is cooled in a fine cooling step, wherein, proceeding from a temperature between 400° C. and 900° C., a cooling rate between 0.1 K/min and 10 K/min is applied.

A method in which, in addition, at least one viscosity modifier or at least one catalyst or mixtures thereof is (are) added in step a) is particularly preferred.

A method is particularly preferred wherein the pre-ceramic Si polymers are selected from the group comprising polysiloxanes, polyorganosiloxanes, polysilsesquioxanes, silicone resins, silicone rubbers, polysilazanes and polycarbosilanes, wherein the organic groups of the Si polymers are selected from saturated, unsaturated, branched, unbranched, ring-form or open-chain groups with 1 to 6 C atoms, aryl, aralkyl or alkylaryl groups with up to 9 C atoms.

Further, a method is particularly preferred in which the glass powder is selected from the group comprising flat glass, window glass, container glass, bottle glass, industrial glass, incandescent bulb glass, television tube glass, laboratory apparatus glass, lead crystal glass, fiber glass, E-glass or borosilicate glass.

Also, a method is particularly preferred wherein the glass converter is selected from the group comprising Na₂CO₃, H₃BO₃, K₂CO₃, Li₂CO₃, CaCO₃, MgCO₃, Al₂O₃ and Na₂B₄O₇, as well as their water-containing derivatives, and from mixtures of these named compounds.

Further, a method is preferred wherein the foams that are produced are infiltrated with a polymer selected from PMMA, PEEK or EVA.

Also, a method is preferred wherein the produced foams are infiltrated by a glass that softens at lower temperatures than the glass foam.

A subject of the invention is thus a method, by means of which: highly light-transparent glass foams and glass-ceramic foams made of filled-polymer, cellular green materials having optical absorption edges lying in the UV region are formed directly from a) a pre-ceramic polymer, b) a glass filler powder and c) one or more components for modifying the glass structure (glass converters), without passing through the roundabout way of a low-viscosity melt phase.

The invention relates to a method which is characterized in that an organosilicon pre-ceramic polymer is used as the SiO₂ source for the production of an optically transparent glass foam or glass-ceramic foam (e.g., polysiloxane of the general composition (R₁R₂SiO_{1.5})ₙ, with R₁, R₂ as organic functional groups, e.g., phenyl, methyl, vinyl or hydrogen, wherein R₁ may also be equal to R₂, polysilsesquioxanes, silicone resins, silicone rubbers, polysilazanes, polycarbosilanes).

Particularly preferred is a method which is characterized in that a glass powder and one or more typical glass converter components, e.g., Na₂CO₃ and its related water-containing derivatives, H₃BO₃ and its water-containing derivatives, K₂CO₃ and its water-containing derivatives, Li₂CO₃, CaCO₃, MgCO₃, Al₂O₃, Na₂B₄O₇*10H₂O or such compounds with a hybrid function as both a glass former and a glass converter, in compositions and concentrations typical for glass formation, are added to the organosilicon polymer.

A method is preferred which is characterized in that an organosilicon monomer, typically a trialkoxyalkylsilane (e.g., trimethoxymethylsilane, triethoxymethylsilane), tetraalkoxysilanes (tetraethoxysilane, TEOS) can be added to adjust the viscosity.

In addition, a method is preferred which is characterized in that a catalyst such as, for example, oleic acid or aluminum acetylacetonate or a combination of both catalysts can be used for accelerated solidification of the batch.

Particularly preferred is a method which is characterized in that the conversion to an open-cell foam is produced directly by heating the batch.

In addition, a method is preferred which is characterized in that a polymer template is coated with the batch containing the essential components and the batch is made to adhere to the template by a liquid-solid transition.

A method is particularly preferred which is characterized in that the organic components introduced via pre-ceramic polymers and/or polymer template are removed oxidatively by a tempering step in air at 350° C. to 700° C.

In addition, a method is preferred which is characterized in that the thermal treatment for glass formation occurs at temperatures lower than or equal to 1100° C.

Particularly preferred is a method which is characterized in that there is a holding step of 1 to 12 hours at maximum temperature.

In addition, a method is preferred which is characterized in that a rapid cooling of the monolithic foam material occurs by removing it from the oven.

A method is particularly preferred which is characterized in that a fine cooling step takes place by transfer into a pre-heated oven having a temperature between 400 and 900° C. and cooling rates are adjusted to between 0.1 and 10 K/min.

In addition, a method is preferred which is characterized in that glass foams produced according to the reticulate method are infiltrated with a polymer (e.g., PMMA, PEEK, EVA).

A method is particularly preferred which is characterized in that glass foams produced according to the reticulate method are infiltrated with a glass that softens at lower temperatures than the glass foam.

Another subject of the present invention is an optically transparent glass foam or glass-ceramic foam that is produced by the method according to the invention.

Another subject of the present invention is the use of the glass foams and glass-ceramic foams for predominantly optical applications, such as, for example, as a support for photocatalysts, a support for enzymes/microbes that operate with light/sunlight in bioreactors, in environmental catalysis, biocatalysis and, for example, for the removal of VOCs from wastewater and air.

### BRIEF DESCRIPTION OF THE FIGURES

FIG. 1 shows a block diagram of the production of an optically transparent glass/glass-ceramic foam;

FIG. 2 shows absorption edges of the glass foams according to the invention.

### DETAILED DESCRIPTION OF THE METHOD ACCORDING TO THE INVENTION

For the production of highly light-transparent foams, the method according to the invention is applied in two different ways:

a) as a direct foaming method (Gambaryan-Roisman, T., Scheffler, M., Buhler, P., Greil, P., Ceram. Trans., 2000, 108, 121-130) and
b) as a special type of reticulate method according to Schwartzwalder (K. Schwartzwalder, A. V. Somers, U.S. Pat. No. 3,090,094, 1963). Both together represent processes of glass formation for obtaining the open-cell foam structure.

A silicon-based pre-ceramic polymer of the polysiloxane, polysilsesquioxane, silicone resin, silicone rubber, polysilazane or polycarbosilane type serves as the SiO₂— supplying initial component, which is contained in the later glass having a composition similar to a filler glass. If this component is aged in air at 800 to 1100° C. (independent of whether it is a foam or a compact material), oxidation of the organic components results along with the volatilization of these components as well as the conversion of the silicon present in the polymer to SiO₂, the later principal glass component. In order to generate a composition that corresponds to that of the glass filler powder with this SiO₂, glass converters must be added. Whereas the ratio of the polymer mass mP to the mass of the glass converter mM is established according to the polymer employed and the composition of the glass filler powder, the mass fraction of the glass-filler powder mG to the sum of mP and mM, however, can be selected variably, if the nominal composition of the total glass shall correspond on average to that of the glass powder. Alternatively, the fraction of glass converter, however, can be selected within broad limits. In this case, the procedure starts by weighing out the components a), b) and c) and homogenizing them by means of a suitable mixer. Subsequently, a foam is formed, wherein process temperatures from room temperature up to a maximum of 320° C. are used, each time depending on the composition and the method variant.

In addition, it is common to both process variants that during/after the foam formation, a thermal process is necessary, combined with a liquid-solid transition, for converting the polymer/filler mixture. This thermal process has several functions:

i. the organic components are removed by oxidation,
ii. SiO₂ is generated for the glass formation with the glass converter/glass converters,
iii. the activation energy/temperature is provided for the glass formation.

Whereas point i. is also necessary for the production of ceramic foams, points ii. and iii. and their combination with the composition or use of components a), b) and c) in connection with the thermal conversion of pre-ceramic polymers are completely novel.

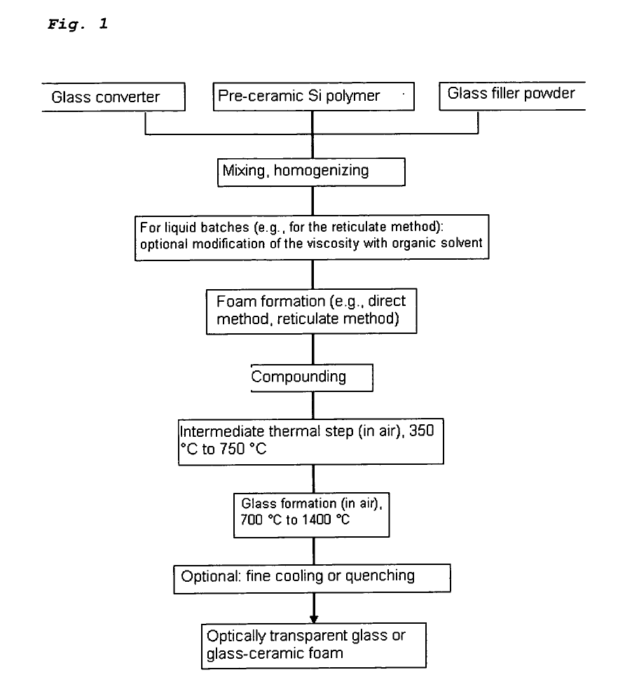

FIG. 1 demonstrates the process for the production of this novel type of optical glass foam according to the present invention.

Novel to the method according to the invention is the fact that the silicon dioxide (SiO₂) phase formed after the oxidation of the organic components from the pre-ceramic polymer (and the polyurethane foam that serves as the cellular template for the reticulate foams) that is used for the foam formation is converted directly into a glass by means of the glass converter for glass properties, which, in its composition, is similar to or the same as the composition of the glass filler. In this way, the sintering process of the glass particles with the newly formed glass is clearly facilitated, so that the thermal processes can be conducted at temperatures up to just 1100° C. (the production of glasses that correspond to the composition of the filler powder used is usually conducted at temperatures up to 1500° C.) and the macrocellular structure of the open-cell foams will be obtained. The method operates independently of whether a so-called green foam (foam after its formation and prior to thermal treatment) is produced according to a direct foaming method or according to a place-holding method (reticulate method, beads that can be fritted, salts). It is essential that the glass-forming SiO₂ component is formed by oxidation at the site and that the glass-converting component is already present and the temperature necessary for sintering with the filler powder can be reduced in comparison to conventional glass production.

The glass and glass-ceramic foams produced according to the invention are excellently suitable as supports for catalysts that can be activated by means of light, so-called photocatalysts. The foams produced according to the invention respond in a special way to the high requirements relative to their transmission of light with the wavelengths that activate the catalyst, but also relative to their permeability for flowing liquids and gases. The products produced according to the invention are monolithic glass and ceramic structures as well as open-cell foams and honeycomb structures.

In the present invention, a method is described that allows the production of a cellular support with good permeability for gases and liquids and a high optical transparency for a large part of the light of wavelengths of the solar spectrum. It preferably involves a glass having a high silicate fraction of conventional composition.

### EMBODIMENT EXAMPLES

### Example 1

60 g of a pre-ceramic polymer (H44, Wacker Siltronic) were weighed out and homogenized in an overhead shaker with 83.5 g of a glass powder of the composition 81% SiO₂, 13.1% B₂O₃, 4% Na₂O/K₂O, 1.9% Al₂O₃ (data in mass %) with a particle size of d₅₀=25 μm, and 8.97 g of Na₂B₄O₇*10H₂O. Subsequently, the batch was foamed in a pre-heated oven at 270° C. (direct foaming by evolution of gases from the components) and aged for 2 hours at this temperature. After cooling, the samples were cut to the desired dimension and heated at 5 K/min to 550° C., kept at this temperature for 4 hours and again heated to 1000° C. After 2 to 4 hours, cooling was conducted by targeted fine cooling or quenching.

### Example 2

77 g of H44 were weighed out and homogenized in the overhead shaker with 59.3 g of a glass powder of the Schott Glas AG company (Duran®) with a particle size of d₅₀=115 μm and 11.9 g of Na₂B₄O₇*10H₂O. Subsequently, the batch was foamed in the preheated oven at 300° C. and aged at this temperature for 4 hours. After cooling, the samples were cut to the desired dimension and heated at 3 K/min to 550° C., kept at this temperature for 2 hours, and again heated to 1000° C. After 2 to 4 hours, cooling was conducted by targeted fine cooling or quenching.

### Example 3

66 g of H44 were weighed out and homogenized in the overhead shaker with 82.7 g of a glass powder of the company Corning, Inc. (Pyrex®) with a particle size of d₅₀=15 μm and 4.7 g of Na₂B₄O₇*10H₂O, 1.25 g of B₂O₃ (which was produced by drying and grounding of H₃BO₃) and 0.95 g of Na₂CO₃. Subsequently, the batch was foamed in the preheated oven at 300° C. and aged at this temperature for 2 hours. After cooling, the samples were cut to the desired dimension and heated at 4 K/min to 550° C., kept at this temperature for 3 hours, and again heated to 1000° C. After 2 to 4 hours, cooling was conducted by targeted fine cooling or quenching.

### Example 4

42.3 g of a methylpolysiloxane (MK, Wacker Siltronic) are dissolved stepwise with 20.1 g of methyltriethoxysilane (MTES, ABCR GmbH) in a glass beaker by means of a magnetic stirrer. 78.5 g of borosilicate glass 3.3 (Schott Glas AG) and 13.4 g of borax are added to the suspension and homogenized. 1.35 g of oleic acid, which is added several minutes prior to the coating of the PU foam, serves as the catalyst for crosslinking. Cut-up pieces of polyurethane foam (dimensions 3 cm*3 cm*5 cm) are immersed in the suspension and subsequently dried in air. Aging is subsequently conducted in a drying cabinet at 70° C., then tempering at 500° C. for 5 hours or 550° C. for 4 hours and heating to 980° C. with an aging time of 3 hours.

### Example 5

As in Example 4, but with repetition of the coating process after drying (coating, drying in air, drying at 70° C.; repeat procedure 3 to 6 times) and subsequent one-time intermediate temperature (500 to 550° C.) and high-temperature treatment.

### Example 6

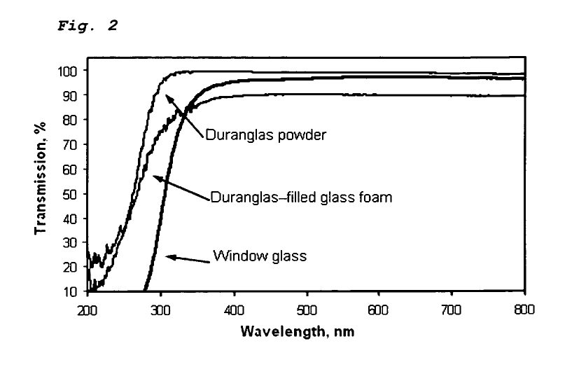

The absorption region was measured in the region of 200 to 800 nm for the glass foam that is filled with Duranglas® and produced according to Example 2. FIG. 2 shows the comparison of the position of the absorption edges of a glass powder (Duran®), a window glass, and a glass foam with Duranglas® of similar composition. It can be seen that the glass foam of the invention according to Example 2 has nearly the same absorption properties as the Duranglas® contained therein.

1. A method for the production of optically transparent foams, wherein the following steps are conducted:
a) Mixing of pre-ceramic Si polymer, glass powder and glass converter;
b) Heating of the mixture to a temperature below the decomposition temperature of the pre-ceramic Si polymer with the formation of a foam;
c) Heating the obtained foam to a temperature above the decomposition temperature of the pre-ceramic Si polymer;
d) Heating the obtained product to a temperature between 700° C. and 1400° C. with the formation of glass; and
e) Cooling the thus-obtained product to ambient temperature.

2. The method according to claim 1, further characterized in that an additional step f) is conducted after step a), comprising:
f) Introducing and/or applying the mixture produced in step a) onto and/or into an organic polymer foam material and subsequently drying the material optionally at elevated temperature.

3. The method according to claim 1, further characterized in that step c) is conducted at a temperature of 350° C. to 750° C.

4. The method according to claim 1, further characterized in that step d) is conducted at a temperature of less than 1100° C.

5. The method according to claim 1, further characterized in that in step d), heating is conducted for 1 to 12 hours at maximum temperature.

6. The method according to claim 1, further characterized in that the product obtained in step e) is rapidly cooled by removal from the oven.

7. The method according to claim 1, further characterized in that the product obtained in step e) is cooled in a fine cooling step, wherein, proceeding from a temperature between 400° C. and 900° C., a cooling rate between 0.1 K/min and 10 K/min is applied.

8. The method according to claim 1, further characterized in that, in addition, at least one viscosity modifier or at least one catalyst or mixtures thereof is added in step a).

9. The method according to claim 1, further characterized in that the pre-ceramic Si polymers are selected from the group comprising polysiloxanes, polyorganosiloxanes, polysilsesquioxanes, silicone resins, silicone rubbers, polysilazanes and polycarbosilanes, wherein the organic residues of the Si polymers are selected from saturated, unsaturated, branched, unbranched, ring-form or open-chain groups with 1 to 6 C atoms, aryl, aralkyl or alkylaryl groups with up to 9 C atoms.

10. The method according to claim 1, further characterized in that the glass powder is selected from the group comprising flat glass, window glass, container glass, bottle glass, industrial glass, incandescent bulb glass, television tube glass, laboratory apparatus glass, lead crystal glass, fiber glass, E-glass or borosilicate glass.

11. The method according to claim 1, further characterized in that the glass converter is selected from the group comprising Na₂CO₃, H₃BO₃, K₂CO₃, Li₂CO₃, CaCO₃, MgCO₃, Al₂O3 and Na₂B₄O₇, as well as their water-containing derivatives, and from mixtures of these named compounds.

12. The method according to claim 2, further characterized in that the foams that are produced are infiltrated with a polymer selected from PMMA, PEEK or EVA.

13. The method according to claim 2, further characterized in that the foams produced are infiltrated with a glass that softens at lower temperatures than the glass foam.

14. An optically transparent glass foam or glass-ceramic foam, produced by the method according to claim 1.

15. A use of the optically transparent glass foam or glass-ceramic foam according to claim 14 for optical applications, as a support for photocatalysts, as a support for enzymes, microbes that operate with light/sunlight in bioreactors, in environmental catalysis, biocatalysis and for removal of VOCs from wastewater and air.
